(19) United States
(12) Patent Application Publication
(10) Pub. No.: US 2018/0028682 A1
(43) Pub. Date: Feb. 1, 2018

(54) MAYTANSINE-DRUG CONJUGATES OF HER-2 SPECIFIC BINDING PROTEINS GENERATED BY SITE SPECIFIC SORTASE-ENZYME MEDIATED CONJUGATION

(71) Applicant: NBE Therapeutics AG, Basel (CH)

(72) Inventors: Ulf Grawunder, Basel (CH); Roger Renzo Beerli, Basel (CH)

(21) Appl. No.: 15/548,659

(22) PCT Filed: Feb. 9, 2016
(86) PCT No.: PCT/EP2016/052742

§ 371 Date: Aug. 3, 2017

Related U.S. Application Data
(60) Provisional application No. 62/113,574, filed on Feb. 9, 2015.

(51) Int. Cl. A61K 47/68

(52) U.S. Cl. CPC A61K 47/6889; A61K 47/6871; A61K 47/6817

(57) ABSTRACT

The present invention relates to a conjugate comprising an anti-HER-2 binding protein site-specifically conjugated to at least one maytansinoid toxic payload by means of sortase enzyme mediated conjugation.

### CROSS-REFERENCE TO RELATED PATENT APPLICATIONS

This application claims the benefit of priority under 35 U.S.C. §371 of International Application No. PCT/EP2016/052742 filed on Feb. 9, 2016 which claims priority to U.S. Provisional Application 62/113,574 filed on Feb. 9, 2016. The content of these earlier filed applications is hereby incorporated by reference in their entirety.

### SEQUENCE LISTING

The present application contains a Sequence Listing that has been submitted in text format via EFS-Web, containing the file name 13318_0028 U1_SequenceListing.txt which is 45,056 bytes in size, created on Aug. 2, 2017, and is hereby incorporated by reference pursuant to 37 C.F.R. §1.52(e)(5).

### FIELD OF THE INVENTION

The present invention discloses novel anti-HER-2 antibody drug conjugates that contain glycine-modified maytansinoid toxin payloads, like maytansine, DM1 or DM4, site-specifically conjugated to the C-termini of either IgH or IgL chains of the antibody by means of sortase mediated antibody conjugation (SMAC)-technology™, such that the toxins are conjugated by strong covalent peptide bonds between the sortase tag, LPXTG (SEQ ID NO: 9), and the glycine tag coupled to the toxin structure. Furthermore, a functional analysis of these SMAC-technology™ conjugated anti-HER-2 maytansinoid ADCs are disclosed, including the efficacy for tumor cell killing in vitro or in vivo in comparison to commercial anti-HER-2 ADC Kadcyla® generated by chemical conjugation.

### Introduction

The therapy of cancer has significantly improved in recent years by the development of novel targeted therapies involving the specific targeting of pharmaceutically active compounds to cancer cells by way of coupling them to binding proteins with highly selective binding to cancer specific targets. The majority of these targeted therapies involve the utilization of antibodies or antibody-derived binding fragments conferring the high selectivity and affinity for a desired cancer cell specific target [Alewine et al. (2015), Wayne et al. (2014), Perez et al. (2014)], although other targeting moieties, like folic acid [Assaraf et al. (2014)] or other scaffold proteins like Fynomers, DARPins, Affibodies and the like that bind to cancer targets with high selectivity may also be employed [Friedman et al. (2009)].

The rationale of the “classical” anti-HER-2 therapy with unmodified antibodies is to suppress HER-2 signalling activity, resulting in inhibition of downstream signalling pathways, cell cycle arrest and a reduction in angiogenesis. Furthermore, antibody binding to the HER-2 extracellular domain may lead to antibody-dependent cell-mediated cytotoxicity (ADCC), and prevents HER-2 receptor extracellular domain cleavage, leading to tumor cell stasis. Furthermore, the administration of an anti-HER-2 antibody may prevent HER-2 from dimerisation with other ligand-activated HER-2 receptors, mostly HER-3.

However, it appears that the majority of HER-2-overexpressing tumors demonstrate primary resistance to single-agent trastuzumab anti-HER-2 antibody. In fact, the rate of primary resistance to single-agent trastuzumab for HER-2-overexpressing metastatic breast cancer is 66% to 88%.

For this reason, new approaches suggest the use of anti-HER-2 antibodies in combination with chemotherapy, or the conjugation of an anti-HER-2 antibody with a toxin (either in the form of so-called immunotoxins, or antibody drug conjugates, see below), where the antibody acts as a targeting device with the function to direct the toxin to the site of a tumor characterized by overexpression of HER-2.

In case of antibody and antibody-fragment based targeting approaches to deliver a pharmaceutically active compound to cancer cells, one needs to distinguish between immunotoxins and antibody drug conjugates, or ADCs [Perez et al. (2014)]. Immunotoxins usually are bacterially produced fusion proteins of antibody binding domain fragments (e.g. single-chain variable domain fragments (scFᵥ)) and highly potent bacterial toxins, like pseudomonas exotoxin, diphtheria or botulinum toxin [Alewine et al. (2015)]. In contrast, ADCs are usually full-length antibodies to which small molecular weight compounds are conjugated by various strategies [Perez et al. (2014)]. While immunotoxins are highly potent for cell killing and only require the internalization of small amounts of bacterial toxins into the cytosol of cancer cells by receptor-mediated endocytosis, a high hurdle for the successful use of immunotoxins for the therapy in humans is the high immunogenicity of the bacterial protein toxin moiety. Therefore, to date not a single immunotoxin molecule has been approved for the therapy of cancer in humans.

In contrast, ADCs, which usually are composed of full-length antibodies and small molecular weight toxic (or pharmaceutically active) compounds, are not associated with major immunogenicity issues in human patients, as long as the antibody moiety itself is non-immunogenic, i.e. either human or humanized. The small molecular weight toxic (or pharmaceutically active) compounds apparently are not sufficiently recognized as immunogenic by the human immune system. As a consequence, the first two ADCs have recently been approved by health authorities for the treatment of human cancer, namely the anti-CD30 targeting ADC brentuximab-vedotin (commercial name Adcetris®), which was FDA approved in 2011 for the treatment of Hodgkin lymphoma, and the anti-HER-2 ADC trastuzumab-emtansine (T-DM1; commercial name Kadcyla®), which was FDA approved in 2013 for the treatment of standard-therapy refractory metastatic breast cancer [Perez et al (2014)]. Based on the high therapeutic success with such novel ADCs, more than 30 ADCs are currently at various stages of clinical trials [Mullard (2013)].

However, the one anti-HER-2 ADC on the market, Kadcyla®, as well as essentially all ADCs in clinical trials, have been manufactured by use of chemical conjugation involving chemical linkers that covalently attach the toxic payload to either primary amino groups of lysine residues in the antibody structure, or to free thiol groups, that are usually generated by mild reduction of intra-chain disulphide bridges of the antibody. The stoichiometry between the antibody and the toxin can thus not entirely be controlled. This results is an inherent heterogeneity of the drug substance, because not all amino or thiol groups are used in the conjugation, resulting in mixtures of ADCs containing from 1 to 8 drugs per antibody (DAR1 to DAR8, DAR meaning: drug-to-antibody ratio), and even containing some non-conjugated antibody (DAR0), which of course does not have any pharmacologic activity, and may even compete with or block the binding of toxin conjugated ADCs [Panowski et al. (2014)]. This again may reduce at least the reproducibility of the therapy as such, if not the efficacy thereof as a whole.

Furthermore, the non-site specific conjugation between the antibody and the toxin may result in a destruction of the binding capacity of at least some of the antibody, e.g., when the conjugation occurs within the variable domain of the antibody.

It is thus the object of the present invention to provide a maytansinoid-comprising conjugate with an anti-HER-2 binding protein that avoids the disadvantages set forth above.

It is another object of the present invention to provide a maytansinoid-comprising conjugate with an anti-HER-2 binding protein that has less side effects and/or can be administered with higher dosages than the anti-HER-2 ADCs from the prior art.

It is another object of the present invention to provide a maytansinoid-comprising conjugate with an anti-HER-2 binding protein that has better stability than the anti-HER-2 ADCs from the prior art, thus avoiding premature release of the maytansinoid in human serum.

It is another object of the present invention to provide a maytansinoid-comprising conjugate with an anti-HER-2 binding protein that has a higher homogeneity of the product as such, with (i) less non-conjugated anti-HER-2 binding protein, (ii) more consistent DAR, and (iii) better site-specificity of the conjugation.

It is another object of the present invention to provide a maytansinoid-comprising conjugate with an anti-HER-2 binding protein in which the binding capacity of the anti-HER-2 binding protein is not affected.

It is another object of the present invention to provide a product comprising a maytansinoid-comprising conjugate with an anti-HER-2 binding protein, with reduced unconjugated anti-HER-2 binding protein, or even devoid of the latter.

It is another object of the present invention to provide a maytansinoid-comprising conjugate with an anti-HER-2 binding protein that has a better reproducibility and/or better efficacy for the therapy.

### PREFERRED EMBODIMENTS

### Brief Description of the Figures

FIG. 1 (a): Amino acid sequences (SEQ ID NOs: 19-22) encoded by the ORF of the expression vectors used to express C-terminally LPETG (SEQ ID NO: 10) sortase-tagged IgH and IgL chains of the HER-2 specific antibodies trastuzumab and FRP-5, as indicated. The disclosed amino acid sequences contain the N-terminal signal peptides (highlighted in boldface print) that are cleaved of in the final IgH and IgL chains of CHO cell expressed antibodies, and the sequences contain the additional C-terminal modifications (highlighted by underline) allowing SMAC-technology™ conjugation of payloads to the C-termini of either IgH or IgL chains. In case of the IgH chains, the C-terminal modification only comprises the LPETG sortase-tag (underline & highlighted in boldface print; (SEQ ID NO: 10)), followed by one glycine residue and an additional strepII affinity tag (WSHPQFEK; (SEQ ID NO: 11)) allowing optional affinity separation of unmodified substrate antibodies. In case of the IgL chains, the C-terminal modification (highlighted by underline) contains an additional GGGGS spacer in front of the LPETG sortase-tag (underline & highlighted in boldface print; (SEQ ID NO: 10)), followed by one glycine residue and an additional strepII affinity tag (WSHPQFEK; (SEQ ID NO: 11)) allowing optional affinity separation of unmodified substrate antibodies. The additional GGGGS spacer, as part of the C-terminal modification of the IgL chains, has been added to facilitate synchronous sortase conjugation of payloads to the antibody IgH and IgL chains.

FIG. 1 (b): Composition of matter of the C-terminally SMAC-technology™ conjugated IgH and IgL chains (Seq ID NOs: 5-8) of the ADCs used in the studies, comprising Gly₅-modified maytansine-toxins (either Gly₅-DM1 or Gly₅-maytansine, as depicted in FIG. 2). The final SMAC-technology™ conjugated anti-HER-2 ADCs, with either the Gly₅-DM1 or Gly₅-maytansine toxin added, lack the signal peptides as well as the terminal glycine of the LPETG sortase tag (SEQ ID NO: 10) and any amino acid sequences thereafter, because the sortase enzyme catalyzed transpeptidation reaction results in cleavage of the peptide bond between the threonine and glycine of the LPETG sortase tag ((SEQ ID NO: 10); leading to the loss of the C-terminal GGWSHPQFEK sequence (SEQ ID NO: 18)) and the formation of a new peptide bond with the N-terminal glycine residue of either the Gly₅-DM1 or Gly₅-maytansine payload).

FIG. 2 (a): Chemical structures of the Gly₅ modified payloads used for SMAC-technology™ conjugation of HER-2 specific antibodies trastuzumab and FRP-5.

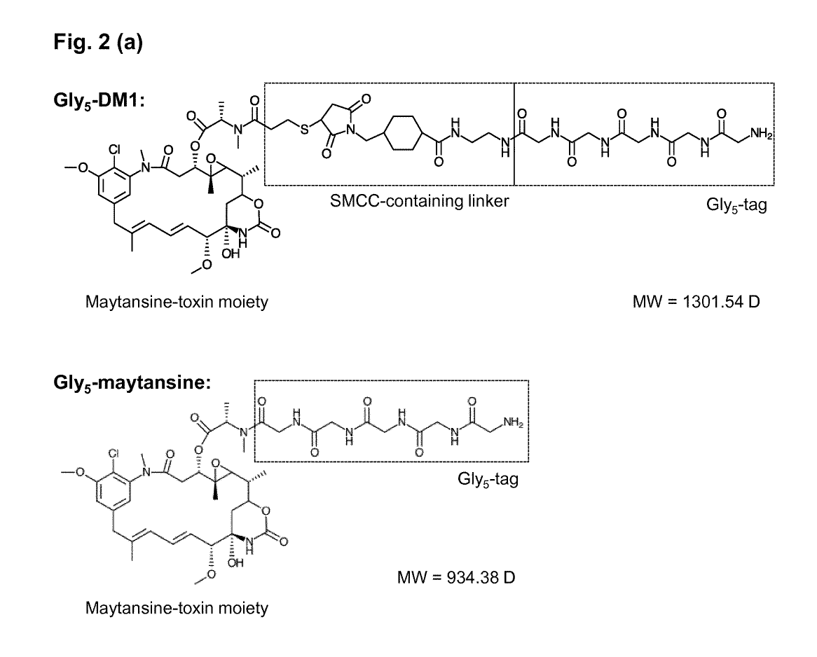

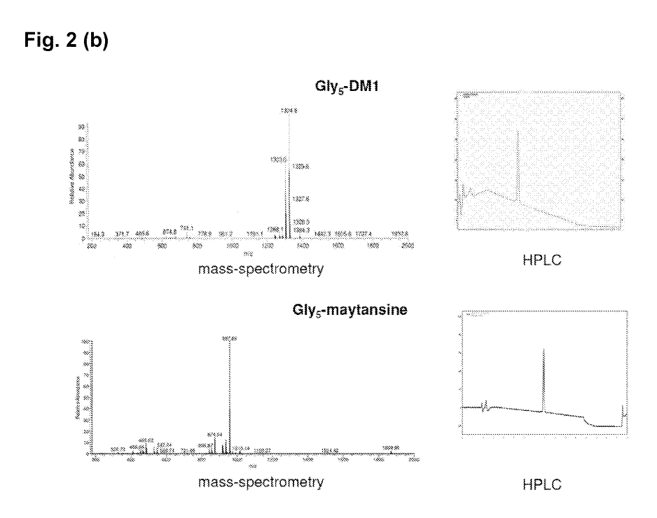

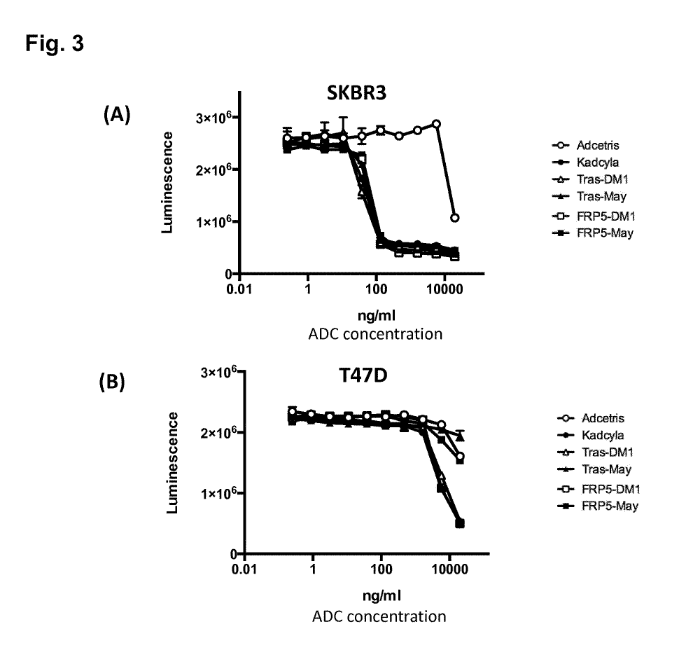

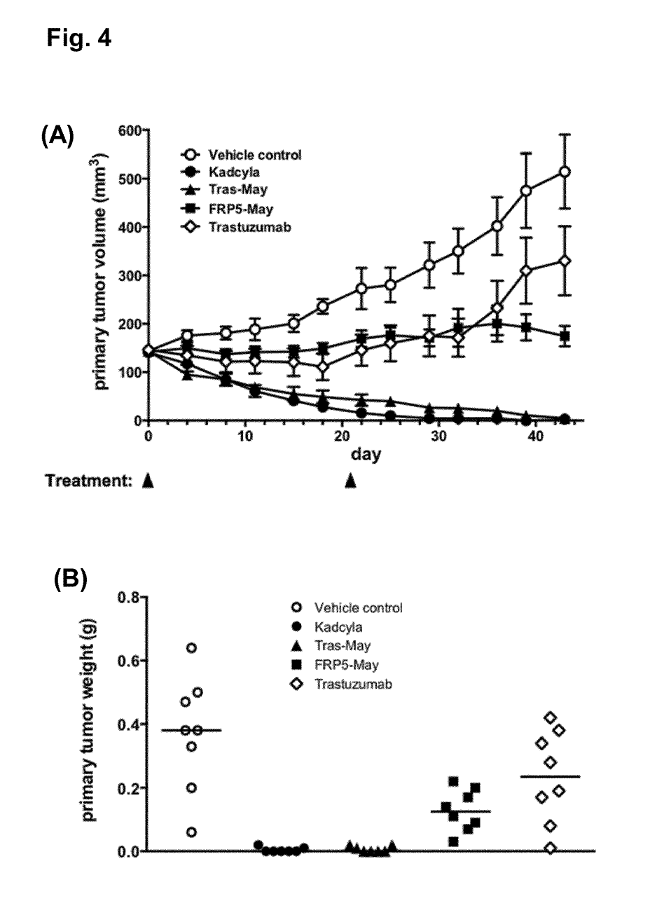

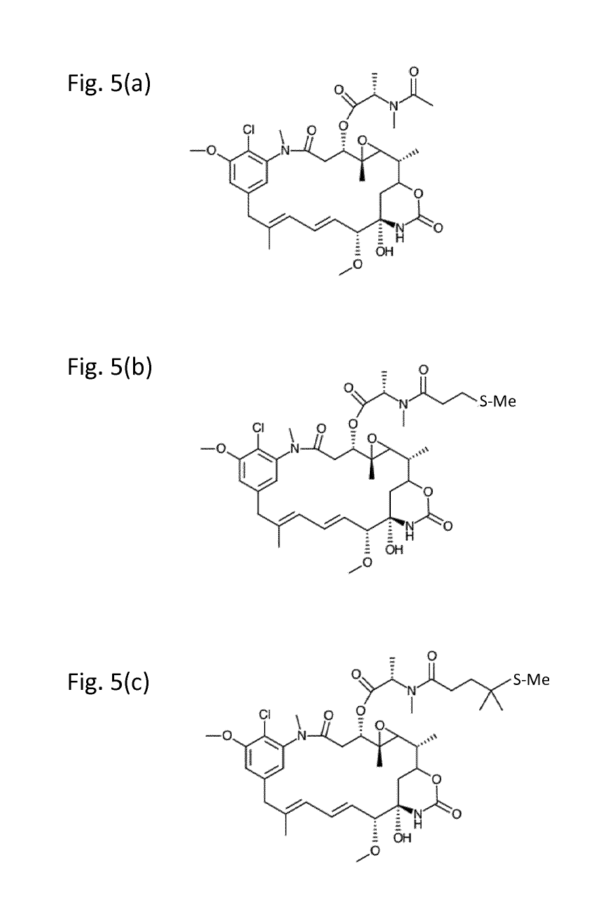

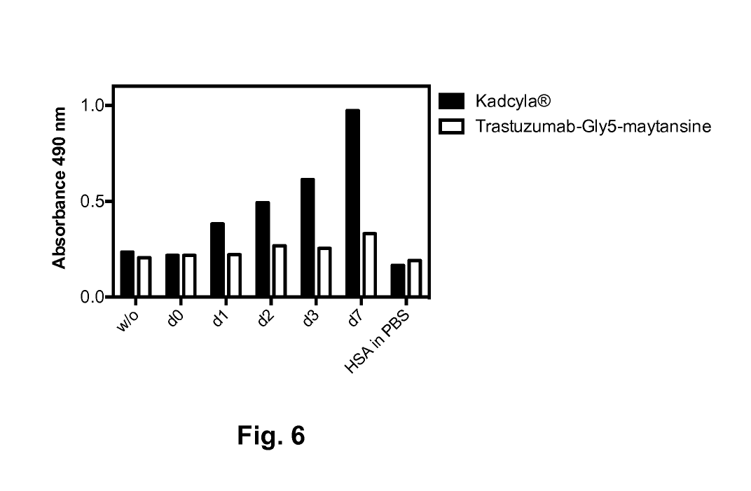

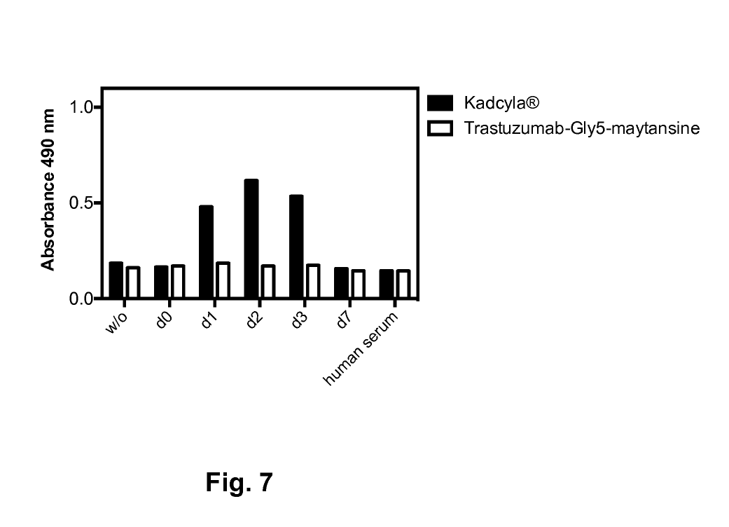

In the upper part of FIG. 2 (a) the maytansinoid is DM1 ([N²′-deacetyl-N²′-(3-mercapto-1-oxopropyl)-maytansine], containing the so-called SMCC linker to which the oligo-glycine peptide (Glyₙ) was coupled, in order to allow conjugation by SMAC-technology™, but to provide the same chemical structure of the DM1 payload in SMAC-technology™ conjugated HER-2 ADCs as in chemically conjugated trastuzumab-DM1. However, this SMCC linker is only an optional component for the SMAC-technology™ conjugated HER-2 ADCs, and of no importance for the conjugation of the payload.

Instead of DM1, other optional linker structures, like the SPDB linker of the maytansinoid payload DM4 ([N20-deacetyl-N20-(4-mercapto-4-methyl-1-oxo-pentyl)-maytansine] may optionally be included, see FIG. 5 (c)

In the lower part of FIG. 2 (a), the maytansinoid is maytansin itself, which in the unconjugated form has the structure of FIG. 5 (a), may be used to generate the oligo-glycine peptide (Glyₙ) derivative depicted here, which has formed the basis for the anti-HER-2 maytansine conjugates analyzed herein.

FIG. 2(b): Analysis of the identity and purity of the Gly₅-DM1 and Gly₅-maytansine payloads of FIG. 2 (a) by mass spectrometry and chromatography, respectively.

FIG. 3: Analysis of HER-2 specific cytotoxic activity of SMAC-technology™ conjugated anti-HER-2-DM1 and anti-HER-2-maytansine ADCs in vitro using human breast cancer cells. Commercially available anti-HER-2 ADC Kadcyla® and anti-CD30 ADC Adcetris® were used as positive and negative controls, respectively. Cytotoxic activity was analyzed on HER-2 overexpressing human breast cancer cells SKBR3 cells (A) and on HER-2^{low} T47D cells (B). Cell viability was quantified using a Luminescent Cell Viability Assay. Datapoints represent mean of two replicates and error bars represent SD.

FIG. 4: In vivo evaluation of HER-2-specific ADCs in a SKOV-3 human ovarian carcinoma mouse xenograft model. SKOV3 human ovarian carcinoma cells were grown subcutaneously in nude mice. Animals were treated i.v. with the indicated ADC (15 mg/kg), Trastuzumab (15 mg/kg) or vehicle control on days 0 and 21. (A) In vivo monitoring of tumor growth until day 43. Data points represent mean and bars represent SEM. (B) Tumor weights determined at necropsy on day 43.

FIG. 5: Three Maytansinoids that can be used in the context of the present invention. FIG. 5 (a): Maytansine, FIG. 5 (b): DM1 ([N²′-deacetyl-N²′-(3-mercapto-1-oxopropyl)-maytansine], FIG. 5 (c): DM4 ([N20-deacetyl-N20-(4-mercapto-4-methyl-1-oxo-pentyl)-maytansine].

FIG. 6: ELISA-based measurement of human serum albumin-bound payload following incubation of each of SMAC-technology™ conjugated trastuzumab-Gly₅-maytansine ADC and Kadcyla® with human serum albumin in PBS over 7 days.

FIG. 7: ELISA-based measurement of human serum albumin-bound payload following incubation of each of SMAC-technology™ conjugated trastuzumab-Gly₅-maytansine ADC and Kadcyla® with human serum over 7 days.

### DETAILED DESCRIPTION OF THE INVENTION

According to a preferred embodiment of the invention, a conjugate comprising an anti-HER-2 binding protein site-specifically conjugated to at least one maytansinoid toxic payload is provided, which conjugation has been carried out by means of sortase enzyme mediated antibody conjugation.

This approach allows to provide a maytansinoid-comprising conjugate with an anti-HER-2 binding protein that has less side effects and/or can be administered with higher dosages than the anti-HER-2 ADCs from the prior art, because, inter alia, premature release of the maytansinoid is reduced. Furthermore, because the stoichiometry between the anti-HER-2 binding protein and the toxin is entirely controlled, the resulting conjugate has a higher homogeneity of the product as such, thus resulting in a better reproducibility of the therapy.

Because the share of unconjugated anti-HER-2 binding protein is reduced or avoided at all, the overall efficacy can be increased, because blocking effects are reduced or avoided. Furthermore, the site specific conjugation avoids that the binding capacity of the anti-HER-2 binding protein is affected.

Thus, we disclose the generation and evaluation of HER-2 specific ADCs for tumor killing in vitro and in vivo that have been generated by a novel, recently developed enzymatic conjugation platform utilizing the bacterial transpeptidase enzyme sortase A from Staphylococcus aureus [WO2014140317A1, incorporated in its entirety herein for the purpose of reference], which is also referred to as SMAC (sortase-mediated antibody conjugation)-technology™.

Because the enzymatic SMAC-technology™ conjugation does not require any chemical linkers, SMAC-technology™ conjugated HER-2 ADCs are disclosed that do not contain any potentially unstable maleimide or other chemical linker structures. Instead, the maytansinoid toxins are conjugated to HER-2 mAbs by sortase mediated transpeptidation between peptide tags required by sortase enzyme for the formation of novel, stable peptide bonds.

This feature offers particular advantages, because it has been shown that some maleimide-linker containing ADCs (which represent >95% of all ADCs in clinical testing) are associated with a specific toxin-transfer to cysteine-34 of human serum albumin (HSA) by a so-called retro Michael reaction (Alley et al. (2008)). Both the fast de-drugging of high DAR species and the transfer of toxins to human serum albumin leads to a significant systemic distribution of toxic payloads from conventional ADCs in patient's circulation, which negatively affects the safety-to-efficacy profile of such ADCs, hence not only increasing toxic side effects thereof, but also reducing efficacy, because receptors are blocked with de-drugged ADCs.

The linker technology as used herein avoids this premature de-drugging, which is shown in comparative experiments against the maleimide-conjugated HER-2 ADC Kadcyla®. The invention have thus provides means to reduce ADC-related side effects plus increase ADC efficacy.

The maytansinoid toxic payload is, in a more general form, discussed as “payload” in WO2014140317A1. WO2014140317A1, which is meant to belong to the disclosed subject matter of the instant application, further provides technical details, disclosure and enablement as regards the sortase conjugation technology, which is also called SMAC-technology™ (sortase mediated antibody conjugation technology) herein.

As used herein, the term “maytansinoid” refers to the toxin maytansine, as disclosed in U.S. Pat. No. 3,896,111, and derivatives thereof. Maytansine is a cytotoxic agent, which inhibits the assembly of microtubules by binding to tubulin at the rhizoxin binding site. It is a macrolide of the ansamycin type and can be isolated from plants of the genus Maytenus.

Preferably, the maytansinoid used in the context of the present invention is maytansine DM1 ([N²′-deacetyl-N²′-(3-mercapto-1-oxopropyl)-maytansine], also called mertansine.

More preferred, said maytansinoid does not comprise the additional SMCC linker structures which it usually is conjugated to. As described herein, the maytansinoid is preferably modified by a glycine tag, useful for transpeptidation reactions using sortase enzymes.

General principles of the sortase technology are disclosed in WO2014140317. This includes the recognition motifs (so called sortase tags) of sortase enzymes. These sortase tags are oligopeptides, usually pentapeptide motifs, which are fused to a first entity (here: the anti-HER-2 binding protein) that is to be conjugated to a second entity (here: the maytansinoid), in such way that the C-terminus of said sortase tags oligopeptides remains free for the conjugation with the second entity.

Such sortase tag is e.g, LPXTG (for sortase A from Staphylococcus aureus; (SEQ ID NO: 9)), with X being any of the 20 naturally occurring amino acids. However, such sortase tags may differ in sequence for sortase enzymes from other bacterial species or for sortase classes, as disclosed in WO2014140317, and in the prior art (see: Spirig et al. (2011)).

In one embodiment, the anti-HER-2 binding protein and the maytansinoid toxic payload are conjugated to one another by means of linker structure X-L₂-L₃-Y, wherein L₂-L₃ represent linkers, and wherein X and Y further represent each one or more optional linkers.

It is important to understand that, with respect to the above linker structure, the maytansionoid would be on the left side and the anti-HER-2 binding protein would be on the right. Optional linker X would thus conjugate L₂ to the maytansinoid, and optional linker Y would conjugate the anti-HER-2 binding protein to L₃. It is important to further understand that in this way of displaying the linker structure, the amino acid sequence of linker L₃ would be shown in C′->N′ orientation.

In this construct, several linkers can form a unitary chain that conjugates one toxin to the one binding protein, and/or several linkers can connect several toxins to the one binding protein. Likewise, the linkers can conjugate two or more subunits of the same binding protein to two or more toxin molecules.

The optional linker X can be any chemical linker structure known in the prior art, that have been used in ADCs to allow specific release of the toxin upon internalization into cancer cells (see e.g. Ducry & Stump (2010) or McCombs et al. (2015).

Some examples for such linkers described in the prior art, which are only provided by way of example and not intended to be limiting, are hydrazone linkers, disulfide linkers, ester linkers, carbamate linkers, oxime linkers or Val-cit-PAB. These linkers are shown schematically below.

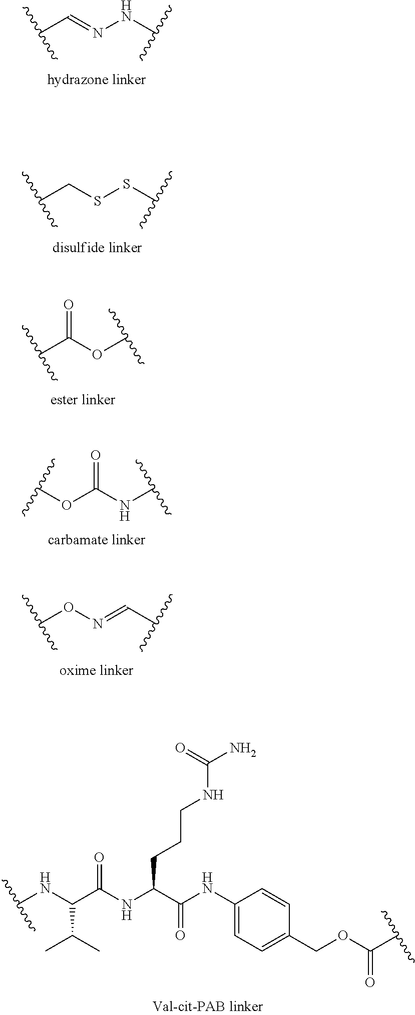

Linkers L₂ and L₃ are discussed below.

The optional linker Y can be any chain of amino acids with up to 20 amino acids allowing optimal conjugation of the anti-HER-2 binding protein to the unitary chain of linkers X, L₂ via L₃.

In another embodiment, the linker structure comprises, as L₂, an oligo-glycine peptide (Glyₙ) coupled to the maytansinoid toxin via its C′-terminus, directly or by means of another linker. Preferably, n in Glyₙ is an integer between ≧1 and ≦21.

In each case (Gly)ₙ (also called (Gly)ₙ-NH₂ or Glyₙ-stretch herein) is a an oligo-glycine peptide-stretch. In one particularly preferred embodiment, n is an integer between 3 and 10, preferably 3 and 6. Most preferred, n=5.

Methods to conjugate the oligo-glycine peptide (Glyₙ) to the maytansinoid toxin are disclosed elsewhere herein.

Optionally, the oligo glycine peptide (Glyₙ) can be conjugated to the maytansinoid toxin by means of a specific linker X.

Such linker is formed e.g., be conjugation of Succinimidyl-4-(N-maleimidomethyl)-cyclohexane-1-carboxylate (SMCC). In the latter case, the use of the linker does not affect the site specificity and the increased stoichiometry of the binding reaction, because the conjugation is effected by the sortase enzym in a site specific and stoichimetric manner, and not by the SMCC linker. Therefore, also any additional linker structure known in the prior art may be included between the maytansine toxin moiety and the oligo-glycin peptide, if a desired linker functionality is deemed to be advantageous. Thus, the advantages set forth above related to the use of the sortase mediated antibody conjugation technology (i.e., less side effects, higher dosages, higher homogeneity of the product as such, reduced blocking effects and unaffected binding capacity of the anti-HER-2 binding protein) are not affected by any optional linker structure that is additionally included.

In another embodiment, the linker structure L₃ comprises a peptide motif that results from specific processing of a sortase enzyme recognition motif during sortase-mediated conjugation.

As disclosed elsewhere herein as well as in WO2014140317, content of which is incorporated by reference herein, sortases (also called sortase transpeptidases) form a group of prokaryotic enzymes that modify surface proteins by recognizing and cleaving a specific sorting signal comprising a particular peptide motif. This peptide motif is also called “sortase enzyme recognition motif”, “sortase tag” or “sortase recognition tag” herein. Usually, a given sortase enzyme has one or more sortase enzyme recognition motifs that are recognized. Sortase enzymes can be naturally occurring, or may have undergone genetic engineering (Doerr et al., 2014).

In another embodiment, the sortase enzyme recognition motif comprises a pentapeptide.

In another embodiment, said sortase enzyme recognition motif comprises at least one of the following amino acid sequences (shown N′->C′):
LPXTG (SEQ ID NO: 9),
LPXSG (SEQ ID NO: 12), and/or
LAXTG (SEQ ID NO: 13).

It is important to understand that in some passages herein, the linker structure is shown as X-L₂-L₃-Y, wherein the maytansinoid would be on the left side and the anti-HER-2 binding protein would be on the right side. In this case, the amino acid sequence of linker L₃ would be shown in C′->N′ orientation.

The first two sortase enzyme recognition motifs are recognized by wild type Staphylococcus aureus sortase A. The second one is also recognized by engineered sortase A 4S9 from Staphylococcus aureus, and the third one is recognized by engineered sortase A 2A-9 from Staphylococcus aureus (Doerr et al, 2014). In all three cases, X can be any of the 20 peptidogenic amino acids.

These sortase enzyme recognition motifs are, for example, fused to the C-terminus of a binding protein, or a domain or subunit thereof, by genetic fusion, and are co-expressed therewith. Said fusion can be done directly, or indirectly, via additional linker Y described elsewhere herein,

It is noteworthy that, once integrated in the linker structure and conjugated to L₂, L₃ lacks the 5th amino acid residue (C-terminal G) of the sortase enzyme recognition motifs. In table 1, said C-terminal G is thus shown in parentheses. In case the sortase enzyme recognition motif is a pentapeptide, L₃ is thus a tetrapeptide.

Prior to sortase conjugation, the sortase enzyme recognition motifs may furthermore carry other tags, like His-tags, Myc-tags or Strep-tags (see FIG. 4a of WO2014140317, the content of which is incorporated by reference herein), fused C-terminal to the sortase enzyme recognition motifs. However, because the peptide bond between the 4th and 5th amino acid of the sortase enzyme recognition motif is cleaved upon sortase mediated conjugation, these additional tags will eventually be removed from the fully conjugated BPDC.

The sortase enzyme recognition motifs can be conjugated to the (Gly)ₙ linker that is coupled to the maytansinoid toxin by means of the sortase technology disclosed herein and in WO2014140317.

It is noteworthy to mention that, while these three peptide stretches are shown above in the classical N-terminus->C-terminus direction, that the L residue is the one that is fused to the C-terminus of the anti-HER-2 binding protein, or to the C-terminus of linker Y, by means of a peptide bond. The 5^{th} amino acid residue (G) of L₃ is removed upon conjugation to the (Gly)n peptide, while the 4^{th} T or S amino acid residue of L₃ is the one that is actually conjugated to the N-terminus of the (Gly)n peptide.

As discussed, the optional linker Y conjugating the pentapeptide recognition motif to the anti-HER-2 binding protein can be any chain of amino acids with up to 20 amino acids allowing optimal conjugation of the anti-HER-2 binding protein to the unitary chain of linkers X, L₂, L₃ or variations thereof, in particular to L₃.

In another preferred embodiment the pentapeptide recognition motif may directly be appended to the last naturally occurring C-terminal amino acid of the immunoglobulin light chains or heavy chains, which in case of the human immunoglobulin kappa light chain is the C-terminal cysteine residue, which in case of the human immunoglobulin lambda light chain is the C-terminal serine residue and which in the case of the human immunoglobulin IgG₁ heavy chain may be the C-terminal lysine residue encoded by human Fcγ1 cDNA. However, another preferred embodiment is also to directly append the sortase pentapeptide motif to the second last C-terminal glycine residue encoded by human Fcγ1 cDNA, because usually terminal lysine residues of antibody heavy chains are clipped off by prosttranslational modification in mammalian cells. Therefore, in more than 90% of the cases naturally occurring human IgG1 lacks the C-terminal lysine residues of the IgG1 heavy chains.

The following a thus gives an overview of the preferred embodiments of the Binding protein-drug conjugate (BPDC) of the invention, with L₂-L₃ shown.

TABLE 1a
Typical linker structures

| | | | L₃ | | |
|---|---|---|---|---|---|
| | | | (shown | | |
| | X | | here | Y | Binding |
| Toxin | (optional) | L₂ | C′ −> N′) | (optional) | protein |
| maytansinoid | e.g., | (Gly)n | (G)TXPL | e.g., amino | anti- |
| | SMCC | | (G)SXPL | acid chain | HER-2 |
| | | | (G)TXAL | (up to 20 | |
| | | | | AA residues) | |

As discussed it is noteworthy that, once integrated in the linker structure and conjugated to L₂, L₃ lacks the 5^{th} amino acid residue (C-terminal G). In table 1, said C-terminal G is thus shown in parentheses.

In the following table, the same molecule is shown, only with a different way display, with the anti-HER-2 binding protein on the left and the maytansionoid on the right. L3 is thus shown in N′->C′.

TABLE 1b
Typical linker structures, displayed in reversed order

| | | L₃ | | | |
|---|---|---|---|---|---|
| | | (shown | | | |
| Binding | Y | here | | X | |
| protein | (optional) | N′ −> C′) | L₂ | (optional) | Toxin |
| anti- | e.g., amino | LPXT(G) | (Gly)n | e.g., | Maytansinoid |
| HER-2 | acid chain | LPXS(G) | | SMCC | |
| | (up to 20 | LAXT(G) | | | |
| | AA residues) | | | | |

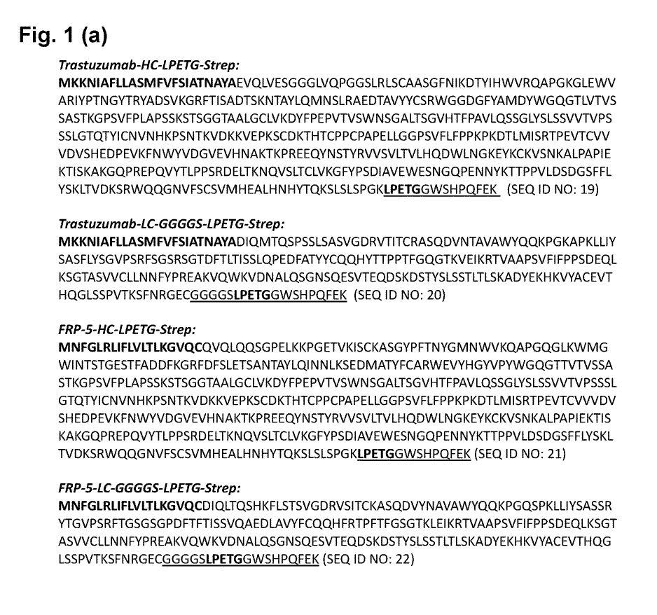

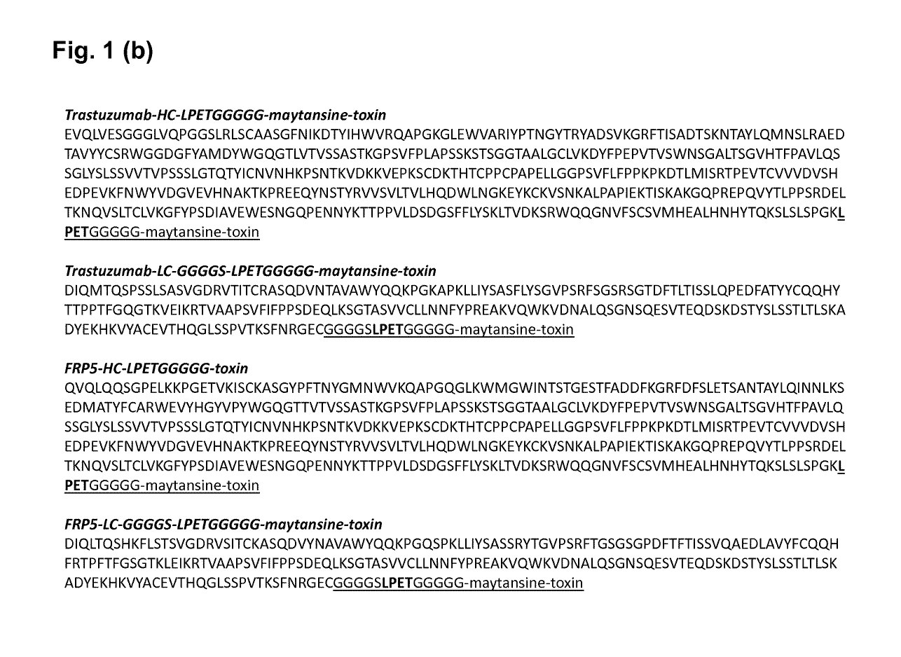

The sortase enzyme is then capable of fusing the two entities to one another by means of a transpeptidation reaction, during which the C-terminal amino acid residue of the sortase tag (e.g., the G in LPXTG is cleaved of, as e.g., shown in FIG. 1 of WO2014140317A1, and then replaced by the first glycine of said Glycine stretch.

Preferably, the anti-HER-2 binding protein is a HER-2 specific antibody.

“Antibodies”, also synonymously called “immunoglobulins” (Ig), are generally comprising four polypeptide chains, two heavy (H) chains and two light (L) chains, and are therefore multimeric proteins, or an equivalent Ig homologue thereof (e.g., a camelid nanobody, which comprises only a heavy chain, single domain antibodies (dAbs) which can be either be derived from a heavy or light chain); including full length functional mutants, variants, or derivatives thereof (including, but not limited to, murine, chimeric, humanized and fully human antibodies, which retain the essential epitope binding features of an Ig molecule, and including dual specific, bispecific, multispecific, and dual variable domain immunoglobulins; Immunoglobulin molecules can be of any class (e.g., IgG, IgE, IgM, IgD, IgA, and IgY), or subclass (e.g., IgG1, IgG2, IgG3, IgG4, IgA1, and IgA2) and allotype.

In such embodiment, the conjugate is an antibody drug conjugate, or ADC. In the following, however, the term “ADC” may also be used for those conjugates in which the anti-HER-2 binding protein is not an antibody in strictu sensu, but an antibody-based binding protein, an antibody derivative or fragment, a modified antibody format, an antibody mimetic, and/or an oligopeptide binder.

According to other preferred embodiments, the anti-HER-2 binding protein is at least one selected from the group consisting of:
antibody-based binding protein
antibody derivative or fragment
modified antibody format,
antibody mimetic, and/or
oligopeptide binder

An “antibody-based binding protein”, as used herein, may represent any protein that contains at least one antibody-derived V_{H}, V_{L}, or C_{H} immunoglobulin domain in the context of other non-immunoglobulin, or non-antibody derived components. Such antibody-based proteins include, but are not limited to (i) F_{c}-fusion proteins of binding proteins, including receptors or receptor components with all or parts of the immunoglobulin C_{H} domains, (ii) binding proteins, in which V_{H} and or V_{L} domains are coupled to alternative molecular scaffolds, or (iii) molecules, in which immunoglobulin V_{H}, and/or V_{L}, and/or C_{H} domains are combined and/or assembled in a fashion not normally found in naturally occurring antibodies or antibody fragments.

An “antibody derivative or fragment”, as used herein, relates to a molecule comprising at least one polypeptide chain derived from an antibody that is not full length, including, but not limited to (i) a Fab fragment, which is a monovalent fragment consisting of the variable light (V_{L}), variable heavy (V_{H}), constant light (CO and constant heavy 1 (C_{H}1) domains; (ii) a F(ab′)2 fragment, which is a bivalent fragment comprising two Fab fragments linked by a disulfide bridge at the hinge region; (iii) a heavy chain portion of a Fab (F_{d}) fragment, which consists of the V_{H} and C_{H}1 domains; (iv) a variable fragment (Fᵥ) fragment, which consists of the V_{L} and V_{H} domains of a single arm of an antibody, (v) a domain antibody (dAb) fragment, which comprises a single variable domain; (vi) an isolated complementarity determining region (CDR); (vii) a single chain Fᵥ Fragment (scFᵥ); (viii) a diabody, which is a bivalent, bispecific antibody in which V_{H} and V_{L} domains are expressed on a single polypeptide chain, but using a linker that is too short to allow for pairing between the two domains on the same chain, thereby forcing the domains to pair with the complementarity domains of another chain and creating two antigen binding sites; and (ix) a linear antibody, which comprises a pair of tandem Fᵥ segments (V_{H}—C_{H}1-V_{H}-C_{H}1) which, together with complementarity light chain polypeptides, form a pair of antigen binding regions; and (x) other non-full length portions of immunoglobulin heavy and/or light chains, or mutants, variants, or derivatives thereof, alone or in any combination.

The term “modified antibody format”, as used herein, encompasses antibody-drug-conjugates, Polyalkylene oxide-modified scFv, Monobodies, Diabodies, Camelid Antibodies, Domain Antibodies, bi- or trispecific antibodies, IgA, or two IgG structures joined by a J chain and a secretory component, shark antibodies, new world primate framework+non-new world primate CDR, IgG4 antibodies with hinge region removed, IgG with two additional binding sites engineered into the CH3 domains, antibodies with altered Fc region to enhance affinity for Fc gamma receptors, dimerized constructs comprising CH3+VL+VH, and the like.

The term “antibody mimetic”, as used herein, refers to proteins not belonging to the immunoglobulin family, and even non-proteins such as aptamers, or synthetic polymers. Some types have an antibody-like beta-sheet structure. Potential advantages of “antibody mimetics” or “alternative scaffolds” over antibodies are better solubility, higher tissue penetration, higher stability towards heat and enzymes, and comparatively low production costs.

Some antibody mimetics can be provided in large libraries, which offer specific binding candidates against every conceivable target. Just like with antibodies, target specific antibody mimetics can be developed by use of High Throughput Screening (HTS) technologies as well as with established display technologies, just like phage display, bacterial display, yeast or mammalian display. Currently developed antibody mimetics encompass, for example, domain ankyrin repeat proteins (called DARPins), C-type lectins, A-domain proteins of S. aureus, transferrins, lipocalins, 10th type III domains of fibronectin, Kunitz domain protease inhibitors, ubiquitin derived binders (called affilins), gamma crystallin derived binders, cysteine knots or knottins, thioredoxin A scaffold based binders, SH-3 domains, stradobodies, “A domains” of membrane receptors stabilised by disulfide bonds and Ca2+, CTLA4-based compounds, Fyn SH3, and aptamers (peptide molecules that bind to a specific target molecules).

The term “oligopeptide binder”, as used herein, relates to oligopeptides that have the capacity to bind, with high affinity, to a given target. The term “oligo” refers to peptides that have between 5 and 50 amino acid residues.

According to a preferred embodiment, the drug-to-binding-protein ratio (DBPR) of the conjugate is anything between 1-8.

A DBPR of 2, for example, means that a binding protein carries two maytansioids.

In a preferred embodiment, the DBPR is a DAR (“Drug-to-Antibody Ratio”). Here, because of the site-specific conjugation of the antibody with the maytansinoid, preferred embodiments comprise that

a) a maytansinoid is conjugated to the C-termini of the heavy or the light chain alone (resulting in ADCs with DAR2, when the antibody is, e.g., an IgG or a F(ab′)₂, or in ADCs with DAR1, when the antibody is, e.g., an scFv fragment), or
b) at both the heavy and the light chain (resulting in ADCs with a DAR4, when the antibody is, e.g., an IgG or a F(ab′)₂, or in ADCs with DAR2, when the antibody is, e.g., a Fab fragment).

Preferably, and by way of example, the anti-HER-2 antibody is Trastuzumab, FRP-5 [Harwerth et al. (1992)], or a derivative or fragment thereof retaining target binding properties. However, other anti-HER-2 antibodies, like for instance, but not limited to Pertuzumab may be used for the invention. The anti-HER-2 monoclonal antibody trastuzumab binds to domain IV of HER-2, while Pertuzumab binds to domain II of HER-2.

Preferably, the antibody comprises the primary amino acid sequences of IgH and IgL chains of FIG. 1 (a) (SEQ ID NOs: 1-4) or of FIG. 1 (b) (SEQ ID NOs: 5-8).

In another preferred embodiment, the maytansinoid toxic payload is at least one selected from the group shown in FIG. 2(a) or FIG. 5.

The preferred maytansinoids are Maytansine itself, or derivatives like DM1 ([N²′-deacetyl-N²′-(3-mercapto-1-oxopropyl)-maytansine]), or DM4 ([N20-deacetyl-N20-(4-mercapto-4-methyl-1-oxo-pentyl)-maytansine]) (FIG. 5 (a), (b), (c), respectively).

Preferably, the oligo-glycine peptide (Glyₙ) is directly coupled to the maytansine core structure or to DM1, as e.g., shown in FIG. 2 (b).

According to an alternative embodiment, the oligo-glycine peptide (Glyₙ) and the maytansinoid toxic payload comprise an optional linker structure X, as e.g., shown in FIG. 2 (a).

Such linker is, e.g., Succinimidyl-4-(N-maleimidomethyl)-cyclohexane-1-carboxylate (SMCC). In the latter case, the use of the linker does not affect the site specificity and the increased stoichiometry of the binding reaction, because the conjugation is effected by the sortase enzyme in a site specific and stoichimetric manner, and not by the SMCC linker. Therefore, also any additional linker structure known in the prior art may be included between the maytansine toxin moiety and the oligo-glycin peptide, if a desired linker functionality is deemed to be advantageous. Thus, the advantages set forth above related to the use of the sortase mediated antibody conjugation technology (i.e., less side effects, higher dosages, higher homogeneity of the product as such, reduced blocking effects and unaffected binding capacity of the anti-HER-2 binding protein) are not affected by any optional linker structure that is additionally included.

Therefore, the invention provides compounds of the following general structure:

M-X-L₂-L₃-Y-BP

in which M is a maytansinoid toxin, L₂ is an oligo-glycine peptide as discussed above, L₃ is a peptide motif that results from specific cleavage of a sortase enzyme recognition motif as discussed above, and BP is an anti-HER-2 binding protein. X and Y further represent each one or more optional linkers as discussed above.

According to another preferred embodiment of the present invention, a method of producing a conjugate according to the above description is provided, wherein an anti-HER-2 binding protein carrying a sortase tag on at least one C-terminus is conjugated, by means of a sortase enzyme, to at least one maytansinoid toxic payload to which an oligo-glycine peptide (Glyₙ) is conjugated. The oligo-glycine peptide (Glyₙ) can be directly coupled to the maytansinoid toxic payload, or via an optional linker.

In this embodiment the oligo-glycine peptide (Glyₙ) has a free N-terminus which is needed for the sortase-mediated reaction as a nucleophile to attack the pepdie bond between amino acid 4 and 5 of the sortase tag. The sortase technology, its advantages (site specific conjugation, stoichimetrically defined relationship between toxin and binding protein, high efficiency of conjugation) is in detail explained in WO2014140317A1. Such sortase tag is, e.g, LPXTG (for Staphylococcus aureus sortase A; (SEQ ID NO: 9)) or LPXTA (for Streptococcus pyogenes sortase A; (SEQ ID NO: 16)), with X being any of the 20 naturally occurring amino acids. According to still another preferred embodiment of the present invention, the use of a conjugate according to the above description for the treatment of a human or animal subject suffering from or being at risk of developing a given pathologic condition is provided.

Alternatively, the invention provides the use of a conjugate according to any the above description for the manufacture of a drug or medicine for the treatment of a human or animal subject suffering from or being at risk of developing a given pathologic condition.

Preferably, said pathologic condition is a neoplastic disease. More preferably, said neoplastic disease is a malignancy, like a tumor, a cancer, or a leukemia.

Even more preferably, said neoplastic disease is a HER-2 positive neoplastic disease.

The term “HER-2 positive” means that the respective tumor exhibits an overexpression of HER-2/erbb2. This overexpression can be diagnosed with methods according to the art, e.g., with the Scoring Hercep Test provided by Dako. This test results in the classification of the tumor into four scores, namely 0, 1+, 2+, and 3+. In the context of the present invention, a HER-2 positive neoplastic disease would be anything from 1+ to 3+, preferably from 2+ to 3+, and most preferably 3+. Most preferably, said neoplastic disease is a breast cancer.

As a summary, it is disclosed that the novel anti-HER-2 ADCs containing maytansinoid toxins directly conjugated to the anti-HER-2 antibody via a glycine-peptide bridge have equivalent potency for in vitro and in vivo killing of human breast and ovarian cancer cells as the chemically conjugated HER-2 ADC Kadcyla® that is currently used for treatment of breast cancer patients. However, the stable peptide bond, by which the maytansinoid toxin payload is attached to the HER-2 antibody is expected to increase the stability of SMAC-technology™ conjugated HER-2 ADCs in human serum, potentially increasing the therapeutic window of such novel ADCs and potentially reducing their side-effect profile in comparison to current HER-2 ADCs manufactured by standard chemical maleimide linker based chemistry.

### FURTHER DESCRIPTION

In order to overcome the main limitations of traditional maleimide linker chemistry for the generation of BPDCs and ADCs, we have previously developed an enzymatic approach for generating BPDCs or ADCs using sequence-specific transpeptidase enzymes, either employing sortase enzymes, or so-called split-inteins (see: WO2014140317A1). In particular, it could be demonstrated that site-specific conjugation of small molecular payloads by sortase enzymes, in the context of antibodies, referred to as SMAC-technology™ (sortase-mediated antibody conjugation technology), results in ADCs that are equally potent as chemically conjugated ADCs in killing cancer cells in vitro. However, first, in SMAC-generated ADCs no maleimide linker chemistry was employed, and second, the conjugation reaction was performed in a site-specific manner to the C-termini of either IgH or IgL chains of the antibody, so that more homogeneous ADCs have been obtained.

In case of SMAC-technology™, the site-specific conjugation can be effected by e.g. recombinant sortase A enzyme of Staph. aureus, that specifically recognizes an LPXTG (SEQ ID NO: 9) pentapeptide (X=any of the 20 naturally occurring amino acids) motif and that can be appended to a recombinant antibody intended for conjugation. Sortase A then uses an oligo-glycine-stretch as a nucleophile to catalyze a transpeptidation, by which the amino group of the oligo-glycine effects a nucleophilic attacks to the peptide bond between the threonine and glycine of the LPXTG (SEQ ID NO: 9) pentapeptide motif. This results in the breakage of that peptide bond and the formation of a new peptide bond between the N-terminal glycine of the oligo-glycine peptide and the fourth amino acid of the sortase tag (the threonine in case of the LPCTG (SEQ ID NO: 17) tag), such that the process results in a transpeptidation.

While it has been shown that trastuzumab-DM1 conjugates generated by sortase-mediated conjugation have comparable potency to the chemically conjugated DM1 conjugates (T-DM1, or Kadcyla®, already applied in the clinic) (Beerli et al. (2015) PLoS One 10, e0131177), higher potency of SMAC-technology generated ADCs has not been achieved (WO2014140317A1). This would not have been expected, because the same targeting antibody and the same payload have been employed.

However, it is surprising that even the complete omission of any linker structure of the maytansinoid payload results in comparable in vitro and in vivo potency for tumor cell killing relative to the chemically conjugated DM1 conjugates (T-DM1, or Kadcyla®, already applied in the clinic).

### EXPERIMENTS AND FIGURES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus.

### Example 1: Production of SMAC-Technology™ Conjugated HER-2-Maytansine ADCs

Sortase A.

Recombinant and affinity purified Sortase A enzyme from Staphylococcus aureus was produced in E. coli as disclosed in WO2014140317A1.

Cloning of Antibody Expression Vectors.

Cloning of expression vectors for the IgH and IgL chains of HER-2 specific antibodies trastuzumab (Goldenberg, 1999) and FRP-5 (Harwerth et al., 1992), encoding a C-terminal extension at the heavy chains (LPETGGWSHPQFEK; (SEQ ID NO: 14)) and the light chains (GGGGSLPETGGWSHPQFEK; (SEQ ID NO: 15)) comprising the LPETG sortase recognition motif (SEQ ID NO: 10), was done as follows: Synthetic genes encoding each of these variable and constant regions for the trastuzumab and FRP-5 antibodies, flanked by suitable restriction sites, were produced by total gene synthesis (GenScript, Piscataway, USA). Expression vectors encoding each of the full-length heavy and light chains were assembled in the proprietary mammalian expression vector pEviS by Evitria AG, Schlieren, Switzerland. The entire amino acid sequences for C-terminally modified IgH and IgL chains for the trastuzumab and FRP-5 based antibodies are disclosed in FIG. 1 (a).

Expression & Purification of Antibodies.

C-terminally modified HER-2 specific antibodies trastuzumab and FRP-5 have been transiently expressed in CHO cells by methods known in the art and recombinant antibodies have been purified by standard protein A purification from CHO cell supernatants as known in the art. In short, the CHO cell supernatants were harvested by centrifugation and sterile filtered (0.2 μm) before FPLC-based affinity purification using Amsphere protein A columns (JSR Life Sciences). Bound antibody was eluted in 0.1M glycine pH 2.5 to 3.5 and immediately neutralized with 1M Tris-HCl buffer at pH 7.5. Buffer exchange to Dulbecco's PBS was done by overnight dialysis.

The purity and the integrity of the recombinant antibodies was analyzed by SDS-PAGE and it was determined that 35.1 and 34.1 mg of recombinant, C-terminally modified trastuzumab and FRP-5 antibody was obtained, respectively, with a purity of >97% with no detectable aggregation or signs of degradation (data not shown).

Generation of Glycine-Modified DM1 and Maytansine Toxins.

In order to generate SMAC-technology™ conjugated HER-2 ADCs with either DM1 or maytansine payloads, pentaglycin modified DM1 and maytansine toxins have been produced by chemistry CRO Concortis, San Diego, U.S.A. The structures of the Gly₅-DM1 and Gly₅-maytansine derivatives are disclosed in FIG. 2 (a). The identity and the purity of the pentaglycine-modified DM1 and maytansine payload was confirmed by mass-spectrometry and HPLC and the results are depicted in FIG. 2(b). From these analyses it can be concluded that each of the Gly₅-modified toxins exhibited >95% purity, as gauged by the single peak in the HPLC chromatogram. The identity of the Gly₅-modified toxins was confirmed by MS analyses and the detected mass for the pentaglycine-modified DM1 of MW=1324.38 D exactly corresponds to the expected mass of MW=1301.54 D plus the MW one Na⁺ ion. Likewise, the detected mass for the pentaglycine-modified maytansine of MW=957.69 D corresponds exactly to the expected mass of MW=934.38 D plus the MW one Na⁺ ion. Therefore, it can be concluded that the toxin derivatives as depicted in FIG. 2(a) have been obtained with the expected structure and at high purity.

Sortase-Mediated Antibody Conjugation.

The above-mentioned toxins were conjugated to antibodies by incubating LPETG (SEQ ID NO: 10)-tagged mAb [10 μM] with Gly₅-modified toxin [200 nM] in the presence of 0.62 μM Sortase A in 50 mM Hepes, 150 mM NaCl, 5 mM CaCl₂, pH 7.5 for 3.5 h at 25° C. The reaction was stopped by passing it through a Protein A HiTrap column (GE Healthcare) equilibrated with 25 mM sodium phosphate pH 7.5, followed by washing with 5 column volumes (CVs) of buffer. Bound conjugate was eluted with 5 column volumes of elution buffer (0.1M succinic acid, pH 2.8) with 1 column volume fractions collected into tubes containing 25% v/v 1M Tris-Base to neutralise the acid. Protein containing fractions were pooled and formulated in 10 mM Sodium Succinate pH 5.0, 100 mg/mL Trehalose, 0.1% % w/v Polysorbate 20 by G25 column chromatography using NAP 25 columns (GE Healthcare) according to the manufacturer's instructions. Following conjugations, the IgH and IgL chains of the trastuzumab and FRP-5 antibodies have the structure as depicted in FIG. 1 (b).

ADC Analytics.

The aggregate content of each conjugate was assessed by chromatography on a TOSOH TSKgel G3000SWXL 7.8 mm×30 cm, 5 μm column run at 0.5 mL/min in 10% IPA, 0.2M Potassium Phosphate, 0.25M Potassium Chloride, pH 6.95. The drug loading was assessed by both Hydrophobic Interaction Chromatography (HIC) and Reverse Phase Chromatography. HIC was performed on a TOSOH Butyl-NPR 4.6 mm×3.5 cm, 2.5 μm column run at 0.8 mL/min with a 12 minute linear gradient between A—1.5M (NH4)2SO4, 25 mM NaPi, pH=6.95±0.05 and B—75% 25 mM NaPi, pH=6.95±0.05, 25% IPA. Reverse phase chromatography was performed on a Polymer Labs PLRP 2.1 mm×5 cm, 5 μm column run at 1 mL/min/80° C. with a 25 minute linear gradient between 0.05% TFA/H2O and 0.04% TFA/CH3CN. Samples were first reduced by incubation with DTT at pH 8.0 at 37° C. for 15 minutes.

The analysis of the drug-to antibody ratio (DAR) determined by Hydrophobic Interaction Chromatography (HIC) and Reverse Phase Chromatography and the percentage of monomeric antibody/ADC as determined by Size-exclusion chromatography is summarized in Table 2 below:

TABLE 2
Analytical summary of conjugates manufactured in this study.

| Conjugate | Target | DAR | Mono (start) | Mono (conj) |
|---|---|---|---|---|
| Trastuzumab-DM1 | HER-2 | 3.05 | 98.1 | 98.1 |
| Trastuzumab-Maytansine | HER-2 | 3.28 | 98.1 | 97.6 |
| cFRP5-DM1 | HER-2 | 3.44 | 98.5 | 97.4 |
| cFRP5-Maytansine | HER-2 | 3.53 | 98.5 | 97.5 |

DAR, drug-to-antibody ratio, determined by hydrophobic interaction and/or reverse phase chromatography; Mono (start/conj), % momomer content before/after conjugation, determined by size exclusion chromatography.

From these analyses it can be concluded that the SMAC-technology™ conjugation has proceeded at high efficiency at each IgH and IgL chain resulting in overall average DARs in the range of ca. 3.0 to 3.5 for each of the antibody-payload combinations. In addition, very low aggregation has been caused by the conjugation of the payloads to the antibodies.

### Example 2: Analysis of the In Vitro Toxicity of SMAC-Technology™ Conjugated, Trastuzumab- and FRP-5-Based Anti-HER-2 ADCs with Gly₅-DM1 or Gly₅-Maytansine Toxin as Toxic Payload

In order to determine the potency of SMAC-technology™ conjugated anti-HER-2 ADCs with either Gly₅-DM1 or Gly₅-maytansine payloads for the HER-2 specific killing of tumor cells, human breast cancer cells lines SKBR3 (overexpressing HER-2 target, data not shown), and T47D (low expression of the HER-2 target, data not shown).

As positive and negative controls, commercially available and thus chemically conjugated ADCs Kadcyla® (anti-HER-2, T-DM1) and Adcetris® (anti-CD30, brentuximab-vedotin) have been used, respectively. For the in vitro breast cancer cell killing assay, cells were plated on 96 well plates in 100 μl growth medium in DMEM/10% FCS at a density of 10′000 cells per well and grown at 37° C. in a humidified incubator at 5% CO₂ atmosphere. After one day culture, 25 μl medium was removed from each well and replaced by 25 μl of 3.5-fold serial dilutions of each ADC in growth medium, resulting in final ADC concentrations ranging from 20 μg/ml to 0.25 ng/ml. Each dilution was done in duplicate. After 3 to 4 additional days, plates were removed from the incubator and equilibrated to room temperature. After approximately 30 minutes, 100 μl CellTiter-Glo® Luminescent Solution (Promega, Cat. No G7570) was added to each well and, after shaking the plates at 450 rpm for 5 min followed by a 10 min incubation without shaking, luminescence was measured on a Tecan Infinity F200 with an integration time of 1 second per well. Luminescence values were plotted against the antibody concentration as depicted in FIG. 3.

Adcetris®, which was used as a negative control, only had a growth-inhibitory effect on SKBR3 cells at the highest concentration of 20 μg/ml, which was even less pronounced in the T47D cells. This level of toxicity at high concentration can be considered as the non-specific toxicity that may be effected on cells not expressing the CD30 target. In contrast, significant tumor cell killing was effected by all anti-HER-2 ADCs, including the SMAC-technology™ conjugated trastuzumab- or FRP-5-based anti-HER-2 ADCs with either Gly₅-DM1 or Gly₅-maytansine payloads. Significantly, the potencies for cell killing between all SMAC-technology™ conjugated anti-HER-2 DM1 and maytansine ADCs were comparable to the positive control and benchmark antibody Kadcyla® (FIG. 3(A)). The IC₅₀ values are summarized in Table 3 below:

TABLE 3
Summary of cell killing activities of anti-HER-2 ADCs
on HER-2-overexpressing SKBR3 breast cancer cells.

| ADC | Target | IC₅₀ |
|---|---|---|
| Kadcyla | HER-2 | 48.4 ng/ml (≈323 pM) |
| Trastuzumab-DM1 | HER-2 | 40.5 ng/ml (≈270 pM) |
| Trastuzumab-Maytansine | HER-2 | 62.9 ng/ml (≈419 pM) |
| cFRP5-DM1 | HER-2 | 67.0 ng/ml (≈447 pM) |
| cFRP5-Maytansine | HER-2 | 77.9 ng/ml (≈519 pM) |

IC₅₀, concentration at which 50% inhibition of cell growth is observed.

In order to confirm that this tumor cell killing activity is specifically effected via the HER-2 target, the same ADCs have also been analyzed for cell killing on HER-2^{low}-expressing T47D human breast cancer cells (FIG. 3 (B)). As expected, only very high concentrations of the five ADCs were able to kill the HER-2^{low} T47D cells, indicating that the toxicity of the ADCs is indeed specific and mediated by HER-2 binding. Therefore, SMAC-technology™ conjugation of Gly₅-DM1 and Gly₅-maytansine to the HER-2 specific mAbs trastuzumab and FRP-5, without any apparent cleavable linker structure, yielded ADCs with in vitro cell killing activities similar to those of the commercially available Kadcyla® manufactured by traditional, chemical conjugation. It is expected that the non-cleavable nature of the Gly₅-maytansine adduct exhibits higher stability and may be dosed higher in patients than a maleimide-linker containing anti-HER-2 ADC.

### Example 3: Analysis of In Vivo Efficacy of SMAC-Technology™ Conjugated Trastuzumab and FRP-5 Based Anti-HER-2 ADCs for Elimination of Human HER-2 Expressing Human Tumor Cells

In order to analyze the potency of SMAC-technology™ conjugated ADCs for HER-2-positive tumor cell killing in vivo, the established SKOV-3 human ovarian carcinoma cell xenograft mouse model has been utilized, and groups of mice with identical number of animals (8) were treated either with SMAC-technology™ conjugated trastuzumab-Gly₅-maytansine ADC, or FRP-5-Gly₅-maytansine ADC, and as positive controls with commercially available Kadcyla® and non-conjugated commercially available trastuzumab (trade-name: Herceptin®). In addition, one group of xenotransplanted mice was only treated with equivalent volumes of vehicle control not containing any antibody or ADC. The experiments have been performed at the qualified contract research organization Proqinase, Freiburg, Germany, which holds all necessary ethical approvals and authorizations for such experiments. The SKOV-3 xenograft mouse model was established by subcutaneous implantation of 5×10⁶ SKOV-3 human ovarian carcinoma cells in 200 μl PBS/Matrigel (1:1). The cells in Matrigel were implanted into the left flanks of NMRI nude mice. Engraftment and growth of the human tumor cells was measured by determining the diameter of the primary tumor volumes by calipering. After 29 days a mean tumor volume of approx. 100-200 mm³ was obtained in the majority of the mice. Tumor-bearing animals were randomized into groups of 8 animals each according to tumor sizes. Animals were treated twice, at day 0 (i.e. at the day of randomization) and 21 days later by intravenous injection of 15 mg/kg ADC, trastuzumab antibody or an equivalent volume of vehicle control. Tumor sizes were monitored twice a week by calipering for a total of 43 days, after which the study was terminated. At the end of the in vivo study, all animals were sacrificed and a necropsy was performed. Primary tumor tissues were collected from all animals and tumor wet weights were determined and recorded. The data for tumor growth or regression is displayed in FIG. 4(A), and the data from the analysis of the wet tumor weight after termination of the study is displayed in FIG. 4(B). As can be seen in FIG. 4(A), as expected, the tumor continuously increased in size to an average size of over 500 mm³ in vehicle control treated mice during the duration of the in vivo study. Treatment of the mice with non-conjugated anti-HER-2 antibody trastuzumab (Herceptin®), that was included as one additional control, resulted in an initial inhibition of further tumor expansion within the first 21 days of the study. However, after second dosing with trastuzumab the ovarian carcinoma started to expand at a similar rate as in vehicle control mice, (FIG. 4 (A)). This suggests that the initial therapeutic effect of the non-conjugated anti-HER-2 antibody trastuzumab has been overcome by the SKOV-3 ovarian carcinoma cells and the cells have become resistant to this therapy. In striking contrast, in animals treated with the commercial anti-HER-2 ADC Kadcyla® the tumor volume regresses and already becomes undetectable between 25-30 days after initiation of treatment (FIG. 4 (A)). Similarly, also treatment of tumor mice with SMAC-technology™ conjugated trastuzumab-Gly₅-maytansine ADC leads to complete regression of the tumor in all mice of the study. Slightly different kinetics of this effect is most likely related to the different concentrations and purities of the ADCs in the SMAC-technology™ conjugated trastuzumab-Gly₅-maytansine ADC versus the commercial Kadcyla®, which as a product that is approved for use in humans is of highest purity and quality. Interestingly, despite the fact that the in vitro potency of FRP-5 and trastuzumab ADCs generated by SMAC-technology™ conjugation has been indistinguishable on SKBR-3 breast cancer cells, treatment of tumor-bearing mice with the FRP-5 based Gly₅-maytansine ADC only led to stabilization of the tumor size during the course of the study, but not to regression of the tumor (FIG. 4 (A)). This underlines that differences in the functional characteristics (e.g. epitope specificity, affinity, intenalization rate, and potentially other functional properties) are of high relevance for the overall potency of an ADC, even if the toxic payload is completely identical.

The analysis of the tumor weight after necropsy (FIG. 4 (B)) is consistent with the tumor growth curves. All mice treated either with commercial Kadcyla®, or with SMAC-technology™ conjugated trastuzumab-Gly₅-maytansine ADC do not show any significant residual tumor masses. Consistent with the measured tumor size, the weights of the tumors at termination of the study show that vehicle control treated mice carry tumors with an average weight of 400 mg, but with quite significant variation. The trastuzumab (Herceptin®) treated mice carry tumors with an average weight of 200 mg, which is somewhat higher than the average weight of the mice treated with FRP-5 based Gly₅-maytansine ADC.

These data demonstrate that the potencies for killing of human SKOV-3 ovarian carcinoma cells in this xenotransplantation model in vivo of commercial Kadcyla® and SMAC-technology™ conjugated trastuzumab-Gly₅-maytansine ADC is comparable. If this is accompanied by an expected higher stability of the SMAC-technology™ conjugated ADC, this may result in a better therapeutic index of the SMAC-technology™ conjugated ADC versus Kadcyla®.

### Example 4: Analysis of In Vitro Transfer of Payload to Human Serum Albumin from SMAC-Technology™ Conjugated Trastuzumab-Based Anti-HER-2 ADC with Gly₅-Maytansine as Compared to Chemically Conjugated Kadcyla® (Anti-HER-2, T-DM1)

The in vitro transfer of payload to human serum albumin from SMAC-technology™ conjugated trastuzumab-Gly₅-maytansine and Kadcyla® ADCs was evaluated in an ELISA-based assay. Briefly, 1 mg/mL of each ADC was diluted 1:1 in PBS (Amimed, 3-05F290-I) containing 50 mg/mL of human serum albumin (Sigma, A3782), or in human serum (Sigma, H6914), and incubated at 37° C. ADC-free PBS with 50 mg/mL of human serum albumin and pure human serum at 37° C. were used as respective background controls. Samples were removed and snap-frozen in liquid nitrogen on days 0, 1, 2, 3, 7 and stored at −80° C. until ELISA analysis. To evaluate maytansine-transfer to albumin, 2 μg/ml of recombinant mouse anti-maytansine antibody (produced in-house) was coated on ELISA plates and blocked with 3% (w/v) of skimmed milk in PBS containing 0.05% (w/v) of Tween-20. The incubated samples were added at a dilution of 1:7500 and bound serum albumin was detected with a 1:1000 dilution of HRP-conjugated rabbit anti-human serum albumin antibody (Abcam, ab60189).

FIG. 6 shows the higher payload transfer to human serum albumin in PBS of maleimide linker-containing Kadcyla® as compared to that of SMAC-technology™ conjugated trastuzumab-Gly₅-maytansine ADC, particularly as of day 1. FIG. 7 shows the higher payload transfer to human serum albumin in human serum of maleimide linker-containing Kadcyla® as compared to that of SMAC-technology™ conjugated trastuzumab-Gly₅-maytansine ADC, particularly as of day 1.

### REFERENCES

Harwerth I M, Wels W, Schlegel J, Müller M, Hynes N E. Monoclonal antibodies directed to the erbB-2 receptor inhibit in vivo tumour cell growth. Br J Cancer. 1993 December; 68(6):1140-5.
Alewine C, Hassan R, Pastan I; Advances in Anticancer Immunotoxin Therapy; Oncologist. 2015 Jan. 5
Alley S C, Benjamin D R, Jeffrey S C, Okeley N M, Meyer D L, Sanderson R J, Senter P D.; Contribution of linker stability to the activities of anticancer immunoconjugates; Bioconjug Chem. 2008 March; 19(3):759-65
Ariazi E A, Ariazi J L, Cordera F, Jordan V C; Estrogen receptors as therapeutic targets in breast cancer; Curr Top Med Chem. 2006; 6(3):181-202.
Assaraf Y G, Leamon C P, Reddy J A; The folate receptor as a rational therapeutic target for personalized cancer treatment; Drug Resist Updat. 2014 October-December; 17(4-6):89-95
Friedman M, Stahl S; Engineered affinity proteins for tumour-targeting applications; Biotechnol Appl Biochem. 2009 May; 53(Pt 1):1-29
Goldenberg M M; Trastuzumab, a recombinant DNA-derived humanized monoclonal antibody, a novel agent for the treatment of metastatic breast cancer; Clin Ther. 1999 February; 21(2):309-18
Ina-Maria Harwerth, Winfried Wels, Barbara M Marte, and Nancy E Hynes; Monoclonal Antibodies against the Extracellular Domain of the erbB-2 Receptor Function as Partial Ligand Agonist; Jul. 25, 1992 The Journal of Biological Chemistry, 267, 15160-15167
Asher Mullard; Maturing antibody-drug conjugate pipeline hits 30; Nature Drug Discovery Volume 12, May 2013
Siler Panowski, Sunil Bhakta, Helga Raab, Paul Polakis and Jagath R Junutula; Site-specific antibody drug conjugates for cancer therapy; mAbs 6:1, 34-45; January/February 2014
Heidi L. Perez, Pina M. Cardarelli, Shrikant Deshpande, Sanjeev Gangwar, Gretchen M. Schroeder, Gregory D. Vite and Robert M. Borzilleri; Antibody-drug conjugates: current status and future directions; Drug Discovery Today Volume 19, Number 7 Jul. 2014
Alan S. Wayne, David J. FitzGerald, Robert J. Kreitman, and Ira Pastan; Immunotoxins for leukemia; Blood, 17 Apr. 2014, Volume 123, Number 16
Spirig et al. (2011), Sortase enzymes in Gram-positive bacteria Mol. Microbiol. 82, 1044-1059).
Dorr B M et al. Reprogramming the specificity of sortase enzymes. PNAS 111:13343-8 (2014).
Ducry L, Stump B., Antibody-drug conjugates: linking cytotoxic payloads to monoclonal antibodies. Bioconjug Chem. 2010 January; 21(1):5-13.
Mc Combs J R, Owen S C Antibody Drug Conjugates: Design and Selection of Linker, Payload and Conjugation Chemistry. The AAPS Journal, March 2015, Volume 17, Issue 2, pp 339-351

1. A conjugate comprising an anti-HER-2 binding protein site-specifically conjugated to at least one maytansinoid toxic payload by means of sortase enzyme mediated conjugation.

2. The conjugate according to claim 1, in which the anti-HER-2 binding protein and the maytansinoid toxic payload are conjugated to one another by means of linker structure X-L₂-L₃-Y, wherein L₂-L₃ represent linkers, and wherein X and Y further represent each one or more optional linkers.

3. The conjugate according to claim 2, wherein the linker structure comprises, as L₂ an oligo-glycine peptide (Glyₙ) coupled to the maytansinoid toxic, directly or by means of another linker, in such a way that the oligo-glycine (Glyₙ) peptide has a free amino terminus, and wherein n is an integer between ≧1 and ≦21.

4. The conjugate according to claim 2, wherein the linker structure L₃ comprises a peptide motif that results from specific cleavage of a sortase enzyme recognition motif.

5. The conjugate according to claim 4, wherein said sortase enzyme recognition motif comprises a pentapeptide.

6. The conjugate according to claim 4, wherein said sortase enzyme recognition motif comprises at least one of the following amino acid sequences
LPXTG (SEQ ID NO: 9);
LPXSG (SEQ ID NO: 12), and/or
LAXTG (SEQ ID NO: 13).

7. The conjugate according to claim 1, wherein the anti-HER-2 binding protein is a HER-2 specific antibody.

8. The conjugate according to claim 1, wherein the anti-HER-2 binding protein is selected from the group consisting of:
antibody-based binding protein
antibody derivative or fragment
modified antibody format,
antibody mimetic, and
oligopeptide binder.

9. The conjugate according to claim 1, wherein the drug-to-binding-protein ratio (DBPR) is anything between 1-8.

10. The conjugate according to claim 1, wherein the antibody is Trastuzumab or FRP-5, or a derivative or fragment thereof which retains target binding capacity.

11. The conjugate according to claim 1, wherein the maytansinoid toxic payload is selected from the group shown in FIG. 2(a) or FIG. 5.

12. The conjugate according to claim 2, wherein the oligo-glycine peptide (Glyₙ) is directly coupled to the maytansinoid toxic payload.

13. The conjugate according to claim 2, wherein the oligo-glycine peptide (Glyₙ) and the maytansinoid toxic payload comprises an optional linker structure.

14. A method of producing a conjugate according to claim 1, wherein an anti-HER-2 binding protein carrying a sortase tag on at least one C-terminus is conjugated, by means of a sortase enzyme, to at least one maytansinoid toxic payload to which an oligo-glycine peptide (Glyₙ) is conjugated.

15. A method of treating a human or animal subject suffering from or being at risk of developing a pathologic condition, the method comprising administering the conjugate of claim 1.

16. The method according to claim 15, wherein said pathologic condition is a neoplastic disease.


SEQUENCE LISTING (22 sequences)

SEQ ID NO: 1 - protein, 487 aa, Artificial Sequence
  Trastuzumab-HC-LPETG-Strep
MKKNIAFLLASMFVFSIATNAYAGVQLVGSGGGLVQPGGSLRLSCAASGFNIKDTYIHWVRQAPGKGLGWVARIYPTNGYTRYADSVKGRFTISADTSKNTAYLQMNSLRAGDTAVYYCSRWGGDGFYAMDYWGQGTLVTVSSASTKGPSVFPLAPSSKSTSGGTAALGCLVKDYFPGPVTVSWNSGALTSGVHTFPAVLQSSGLYSLSSVVTVPSSSLGTQTYICNVNHKPSNTKVDKKVGPKSCDKTHTCPPCPAPGLLGGPSVFLFPPKPKDTLMISRTPGVTCVVVDVSHGDPGVKFNWYVDGVGVHNAKTKPRGGQYNSTYRVVSVLTVLHQDWLNGKGYKCKVSNKALPAPIGKTISKAKGQPRGPQVYTLPPSRDGLTKNQVSLTCLVKGFYPSDIAVGWGSNGQPGNNYKTTPPVLDSDGSFFLYSKLTVDKSRWQQGNVFSCSVMHGALHNHYTQKSLSLSPGKLPGTGGWSHPQFGK

SEQ ID NO: 2 - protein, 256 aa, Artificial Sequence
  Trastuzumab-LC-GGGGS-LPETG-Strep
MKKNIAFLLASMFVFSIATNAYADIQMTQSPSSLSASVGDRVTITCRASQDVNTAVAWYQQKPGKAPKLLIYSASFLYSGVPSRFSGSRSGTDFTLTISSLQPGDFATYYCQQHYTTPPTFGQGTKVGIKRTVAAPSVFIFPPSDGQLKSGTASVVCLLNNFYPRGAKVQWKVDNALQSGNSQGSVTGQDSKDSTYSLSSTLTLSKADYGKHKVYACGVTHQGLSSPVTKSFNRGGCGGGGSLPGTGGWSHPQFGK

SEQ ID NO: 3 - protein, 482 aa, Artificial Sequence
  FRP-5-HC-LPETG-Strep
MNFGLRLIFLVLTLKGVQCQVQLQQSGPGLKKPGGTVKISCKASGYPFTNYGMNWVKQAPGQGLKWMGWINTSTGGSTFADDFKGRFDFSLGTSANTAYLQINNLKSGDMATYFCARWGVYHGYVPYWGQGTTVTVSSASTKGPSVFPLAPSSKSTSGGTAALGCLVKDYFPGPVTVSWNSGALTSGVHTFPAVLQSSGLYSLSSVVTVPSSSLGTQTYICNVNHKPSNTKVDKKVGPKSCDKTHTCPPCPAPGLLGGPSVFLFPPKPKDTLMISRTPGVTCVVVDVSHGDPGVKFNWYVDGVGVHNAKTKPRGGQYNSTYRVVSVLTVLHQDWLNGKGYKCKVSNKALPAPIGKTISKAKGQPRGPQVYTLPPSRDGLTKNQVSLTCLVKGFYPSDIAVGWGSNGQPGNNYKTTPPVLDSDGSFFLYSKLTVDKSRWQQGNVFSCSVMHGALHNHYTQKSLSLSPGKLPGTGGWSHPQFGK

SEQ ID NO: 4 - protein, 252 aa, Artificial Sequence
  FRP-5-LC-GGGGS-LPETG-Strep
MNFGLRLIFLVLTLKGVQCDIQLTQSHKFLSTSVGDRVSITCKASQDVYNAVAWYQQKPGQSPKLLIYSASSRYTGVPSRFTGSGSGPDFTFTISSVQAGDLAVYFCQQHFRTPFTFGSGTKLGIKRTVAAPSVFIFPPSDGQLKSGTASVVCLLNNFYPRGAKVQWKVDNALQSGNSQGSVTGQDSKDSTYSLSSTLTLSKADYGKHKVYACGVTHQGLSSPVTKSFNRGGCGGGGSLPGTGGWSHPQFGK

SEQ ID NO: 5 - protein, 459 aa, Artificial Sequence
  Trastuzumab-HC-LPETGGGGG-maytansine-toxin
GVQLVGSGGGLVQPGGSLRLSCAASGFNIKDTYIHWVRQAPGKGLGWVARIYPTNGYTRYADSVKGRFTISADTSKNTAYLQMNSLRAGDTAVYYCSRWGGDGFYAMDYWGQGTLVTVSSASTKGPSVFPLAPSSKSTSGGTAALGCLVKDYFPGPVTVSWNSGALTSGVHTFPAVLQSSGLYSLSSVVTVPSSSLGTQTYICNVNHKPSNTKVDKKVGPKSCDKTHTCPPCPAPGLLGGPSVFLFPPKPKDTLMISRTPGVTCVVVDVSHGDPGVKFNWYVDGVGVHNAKTKPRGGQYNSTYRVVSVLTVLHQDWLNGKGYKCKVSNKALPAPIGKTISKAKGQPRGPQVYTLPPSRDGLTKNQVSLTCLVKGFYPSDIAVGWGSNGQPGNNYKTTPPVLDSDGSFFLYSKLTVDKSRWQQGNVFSCSVMHGALHNHYTQKSLSLSPGKLPGTGGGGG

SEQ ID NO: 6 - protein, 228 aa, Artificial Sequence
  Trastuzumab-LC-GGGGS-LPETGGGGG-maytansine-toxin
DIQMTQSPSSLSASVGDRVTITCRASQDVNTAVAWYQQKPGKAPKLLIYSASFLYSGVPSRFSGSRSGTDFTLTISSLQPGDFATYYCQQHYTTPPTFGQGTKVGIKRTVAAPSVFIFPPSDGQLKSGTASVVCLLNNFYPRGAKVQWKVDNALQSGNSQGSVTGQDSKDSTYSLSSTLTLSKADYGKHKVYACGVTHQGLSSPVTKSFNRGGCGGGGSLPGTGGGGG

SEQ ID NO: 7 - protein, 458 aa, Artificial Sequence
  FRP5-HC-LPETGGGGG-toxin
QVQLQQSGPGLKKPGGTVKISCKASGYPFTNYGMNWVKQAPGQGLKWMGWINTSTGGSTFADDFKGRFDFSLGTSANTAYLQINNLKSGDMATYFCARWGVYHGYVPYWGQGTTVTVSSASTKGPSVFPLAPSSKSTSGGTAALGCLVKDYFPGPVTVSWNSGALTSGVHTFPAVLQSSGLYSLSSVVTVPSSSLGTQTYICNVNHKPSNTKVDKKVGPKSCDKTHTCPPCPAPGLLGGPSVFLFPPKPKDTLMISRTPGVTCVVVDVSHGDPGVKFNWYVDGVGVHNAKTKPRGGQYNSTYRVVSVLTVLHQDWLNGKGYKCKVSNKALPAPIGKTISKAKGQPRGPQVYTLPPSRDGLTKNQVSLTCLVKGFYPSDIAVGWGSNGQPGNNYKTTPPVLDSDGSFFLYSKLTVDKSRWQQGNVFSCSVMHGALHNHYTQKSLSLSPGKLPGTGGGGG

SEQ ID NO: 8 - protein, 288 aa, Artificial Sequence
  FRP5-LC-GGGGS-LPETGGGGG-maytansine-toxin
DIQLTQSHKFLSTSVGDRVSITCKASQDVYNAVAWYQQKPGQSPKLLIYSASSRYTGVPSRFTGSGSGPDFTFTISSVQAGDLAVYFCQQHFRTPFTFGSGTKLGIKRTVAAPSVFIFPPSDGQLKSGTASVVCLLNNFYPRGAKVQWKVDNALQSGNSQGSVTGQDSKDSTYSLSSTLTSDGQLKSGTASVVCLLNNFYPRGAKVQWKVDNALQSGNSQGSVTGQDSKDSTYSLSSTLTLSKADYGKHKVYACGVTHQGLSSPVTKSFNRGGCGGGGSLPGTGGGGG

SEQ ID NO: 9 - protein, 5 aa, Artificial Sequence
  sortase tag
  misc_feature (3)..(3): Xaa can be any naturally occurring amino acid
LPXTG

SEQ ID NO: 10 - protein, 5 aa, Artificial Sequence
  sortase tag
LPETG

SEQ ID NO: 11 - protein, 8 aa, Artificial Sequence
  strep II affinity tag
WSHPQFEK

SEQ ID NO: 12 - protein, 5 aa, Artificial Sequence
  sortase tag
  misc_feature (3)..(3): Xaa can be any naturally occurring amino acid
LPXSG

SEQ ID NO: 13 - protein, 5 aa, Artificial Sequence
  sortase tag
  misc_feature (3)..(3): Xaa can be any naturally occurring amino acid
LAXTG

SEQ ID NO: 14 - protein, 14 aa, Artificial Sequence
  C-terminal extension at the heavy chain
LPETGGWSHPQFEK

SEQ ID NO: 15 - protein, 19 aa, Artificial Sequence
  C-terminal extension at the light chain
GGGGSLPETGGWSHPQFEK

SEQ ID NO: 16 - protein, 5 aa, Artificial Sequence
  sortase tag
  misc_feature (3)..(3): Xaa can be any naturally occurring amino acid
LPXTA

SEQ ID NO: 17 - protein, 5 aa, Artificial Sequence
  sortase tag
LPCTG

SEQ ID NO: 18 - protein, 10 aa, Artificial Sequence
  Synthetic sequence
GGWSHPQFEK

SEQ ID NO: 19 - protein, 487 aa, Artificial Sequence
  Trastuzumab-HC-LPETG-Strep
MKKNIAFLLASMFVFSIATNAYAEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLPETGGWSHPQFEK

SEQ ID NO: 20 - protein, 256 aa, Artificial Sequence
  Trastuzumab-LC-GGGGS-LPETG-Strep
MKKNIAFLLASMFVFSIATNAYADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSLPETGGWSHPQFEK

SEQ ID NO: 21 - protein, 482 aa, Artificial Sequence
  FRP-5-HC-LPETG-Strep
MNFGLRLIFLVLTLKGVQCQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLPETGGWSHPQFEK

SEQ ID NO: 22 - protein, 252 aa, Artificial Sequence
  FRP-5-LC-GGGGS-LPETG-Strep
MNFGLRLIFLVLTLKGVQCDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSLPETGGWSHPQFEK